(19) United States
(12) Patent Application Publication
(10) Pub. No.: US 2011/0033406 A1
(43) Pub. Date: Feb. 10, 2011

(54) PRESERVED PRODUCT; AND PRESERVATIVE COMPOSITION

(76) Inventors: Peter Finan, Haworth Yorkshire (GB); John Michael Story, Doncaster Yorkshire (GB)

Correspondence Address: NIXON & VANDERHYE, PC, 901 NORTH GLEBE ROAD, 11TH FLOOR, ARLINGTON, VA, 22203, US

(21) Appl. No.: 12/735,722

(22) PCT Filed: Feb. 26, 2009
(86) PCT No.: PCT/GB2009/050194

§ 371 Date: Sep. 27, 2010

(30) Foreign Application Priority Data
Feb. 26, 2008 (GB) 0803473.8

(51) Int. Cl. A61K 8/49; A61K 31/352; A61P 31/04; A61Q 5/00; A61Q 5/12; C11D 3/60; A23L 3/34

(52) U.S. Cl. 424/70.1; 514/456; 510/119; 510/131; 510/319; 426/532

(57) ABSTRACT

A preservative composition held under acidic conditions comprises at least one flavonoid and at least one antimicrobial auxiliary. It is useful as a constituent of a product to be preserved, such as a shampoo, hair conditioner, skin lotion, baby bath wash, moisturiser, medicinal cream, cosmetic product, laundry formulation, paint, oral care product or food preparation.

The present invention relates to a preservative composition, particularly for use in products which may be stored for a long time before use; and a preserved product.

There is a continual demand from formulators, but ultimately from consumers, to have components for use in personal care products (among other products) which have a good environmental profile, are safe to the user, are effective in enhancing preservation, and are compatible in a range of personal care formulations. Present products do not meet all of these requirements.

The object of the present invention is to provide preservative compositions, and formulated products containing said preservative compositions, which offer improvement over existing products, in terms of at least one of the characteristics mentioned above.

According to a first aspect of the present invention there is provided a preservative composition to be added to a formulated product, the preservative composition comprising at least one flavonoid, and at least one antimicrobial auxiliary, wherein the preservative composition is acidic.

Flavonoids are a sub-class of polyphenols, and may include flavonols (eg. quercetin), flavonons (eg. naringin, hesperidin) and/or flavons (eg. rutin).

An antimicrobial auxiliary is a component which acts as an antimicrobial agent or acts as a promoter of enhanced antimicrobial action. That is, it may increase the antimicrobial action of the flavonoid.

An antimicrobial auxiliary may be a suitable surfactant. One suitable surfactant is a betaine having the general structure:

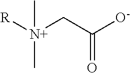

where R is a branched or unbranched alkyl group, optionally substituted, but preferably unsubstituted. Preferably R is an alkyl group having from 4 to 24 carbon atoms, preferably 8 to 20, most preferably 10 to 16. Lauryl betaine (LAB) is an especially preferred surfactant.

A betaine may comprise between 0.1 and 20 wt % of the total composition of the first aspect, and preferably comprises between 2 and 5 wt %.

One suitable antimicrobial auxiliary is a compound which combats moulds or yeasts. Such a compound may comprise sorbic acid and/or a salt thereof, for example potassium sorbate. It may be desirable to provide a compound which combats mould or yeast, for example potassium sorbate or sorbic acid, separately from the composition of the first aspect as defined above; to be combined in the formulation of the end product.

Preferably the composition of the first aspect is miscible with water.

The composition of the first aspect is preferably a liquid. It may be a gel or a viscous cream. Preferably the composition of the first aspect comprises a solvent.

By describing the composition of the first aspect as acidic we mean that it may itself be acidic or may be a precursor of an acidic treatment composition.

The composition of the first aspect may be rendered acidic by addition of organic acid(s), selected from, for example, ascorbic acid, citric acid, lactic acid and malic acid.

The pH of preferred compositions of the first aspect is preferably less than 6, preferably less than 5, preferably between 2 and 4, preferably between pH 2 and 3, and most preferably between 2.1 and 2.6.

The composition of the first aspect is formulated for addition to an end product, for example a personal product such as a shampoo, hair conditioner, skin lotion, baby bath wash, moisturiser, oral care product, medicinal cream or cosmetic product; or a laundry formulation, paint, food preparation for humans, or animal feed; in which products the components of the composition inhibit the growth of undesired microorganisms.

In accordance with a second aspect of the invention there is provided a product to be preserved, the product comprising base components applicable to that product and preservative components comprising at least one flavonoid held under acidic conditions and an antimicrobial auxiliary. The composition of the first aspect may be added together in the form of a precursor formulation or one or more of the components could be added separately (e.g. a sorbate or sorbic acid, when present). The product may be a product as described above, for example a personal care product.

Preferably the product of the second aspect contains from 0.00001 wt % up to 10 wt % bioflavonoid(s) (in total when there is more than one), preferably from 0.00005 wt % up to 5 wt % bioflavonoid(s), preferably from 0.0001 wt % up to 1 wt % bioflavonoid(s), preferably from 0.0002 wt % up to 0.5 wt % bioflavonoid(s). When a mixture of bioflavonoids is employed preferred ranges may be stated as from 0.00001 wt % up to 1 wt % bioflavonoids (in total), preferably from 0.00005 wt % up to 0.1 wt % bioflavonoids, preferably from 0.0001 wt % up to 0.01 wt % bioflavonoids, preferably from 0.0002 wt % up to 0.004 wt % bioflavonoids. The concentration of bioflavonoid(s) in the composition of the first aspect must simply be sufficient to achieve the required amount in the end product.

Preferably the product of the second aspect contains from 0.001 wt % up to 10 wt % of an antimicrobial auxiliary (in total when there is more than one), preferably from 0.01 wt % up to 6 wt % of an antimicrobial auxiliary.

A betaine as an antimicrobial auxiliary is preferably present in an amount from 0.1 wt % up to 10 wt % (in total when there is more than one), preferably from 1 wt % up to 6 wt %.

Sorbic acid and/or a salt thereof as an antimicrobial auxiliary is preferably present in an amount from 0.001 wt % up to 10 wt %, preferably from 0.01 wt % up to 5 wt % (in total when there is more than one such compound).

Preferably the composition of the first aspect, used in a product of the second aspect, prevents the formation of colonies of at least one of these microbes: Pseudomonas aeruginosa, Escherichia coli, Aspergillus niger and Candida albicans. Preferably it prevents the formation of colonies in at least two thereof, preferably at least three thereof, and most preferably all four, under the test methods later described in the Examples. Such a product without the composition of the first aspect does not prevent the formation of colonies which was/were thereby inhibited.

Typically personal care compositions are acidic, often having pH in the range 3-6.5. These are preferred for use in the present invention. The antimicrobial activity of this invention is believed to be more pronounced under acidic conditions. The applicant believes that the equilibrium between quinone/phenolic forms of the flavonoids is shifted favourably under acidic conditions so as to give the form which exhibits the highest antibacterial activity.

A single flavonoid may be present, preferably selected from a flavonol (e.g. quercetin), a flavonon (e.g. naringin, hesperidin) and a flavon (e.g. rutin). The composition of the first aspect preferably comprises at least two flavonoids, preferably at least three flavonoids, preferably selected from a flavonol (e.g. quercetin) and a flavonon (e.g. naringin, hesperidin); and preferably also a flavon (e.g. rutin).

The flavonoid(s) is/are preferably obtained from citrus fruits.

The composition of the first aspect and/or the product of the second aspect preferably comprises at least one organic acid, which may be one or more of ascorbic acid, citric acid, lactic acid and malic acid. Citric acid is particularly preferred. Lactic acid is particularly preferred. Preferably the pH of the composition is kept within the desired range through the addition of lactic acid, most preferably L(+) lactic acid. Citric acid and lactic acid are advantageously used in combination.

The at least one organic acid preferably comprises (in total when there is more than one) from 0.01 wt % to 5 wt % of the product of the second aspect, preferably from 0.05 wt % to 2 wt %, preferably from 0.1 wt % to 1 wt %.

According to a third aspect of the present invention there is provided a method of manufacturing the product of the second aspect, comprising: blending at least one flavonoid, held under acidic conditions, and an antimicrobial auxiliary, into base components of said product.

According to a fourth aspect of the present invention there is provided a method of preventing the formation of undesired microorganisms within a formulated product, by the means defined herein.

Bacteria to be combated may include Pseudomonas aeruginosa and Staphylococcus aureus.

Yeasts and moulds to be combated may include Candida albicans and Aspergillus niger.

Other bacteria which may be combated may include Salmonella typhimurium, Lactobacillus brevis, Escherichia coli, Enterococcus hirae, Enterobacter cloacae, Listeria monocytogenes, Bacillus cereus, Campylobacter Jejuni, MRSA, Clostridia, C. difficile.

Preferred products of the second aspect have antimicrobial activity (colony count reduction) against at least one of Pseudomonas aeruginosa, Staphylococcus aureus, Candida albicans and Aspergillus niger of at least 10² within 7 days of challenge under the test conditions described in the Examples, and preferably show no recovery therefrom in the following 21 days; and preferably have such antimicrobial activity against at least two of them, preferably against at least three of them, most preferably against all four of them.

According to a fifth aspect of the present invention there is provided the use of a composition of the first aspect, or of its components separately, to impart antimicrobial activity to a formulated product.

Any preferred feature of any aspect of the invention expressed herein is a feature of the invention as a whole and is to be regarded as preferred feature of any other aspect.

When amounts of components of a composition are expressed they denote the percentage weight of the stated component as a percentage of the total composition weight, unless otherwise stated.

The following examples are not limiting. They are given only by way of illustration of the practice of the invention.

### EXAMPLES

In these examples all ingredient quantities are quoted as wt % of the total composition, and relate to undiluted individual ingredients, except for:
lactic acid, which is an 80 wt % aqueous solution of L(+) lactic acid sold under the trade name Purac FCC80 or Purac Sanilac. The amounts stated in these examples refer to the as-supplied material.
mixed bioflavonoids (hereinafter MBF) is a flavonoids-containing component containing including flavonols (eg. quercetin), flavonons (eg. naringin, hesperidin) and/or flavons (rutin). The mixed bioflavonoids component contains 0.4% of said flavonoids. The amount stated refers to the as-supplied material.
decyl glucoside which is a 50% aqueous solution. The amount stated refers to the as-supplied material; and
the betaine composition, which is a formulation of lauryl betaine, as a 30 wt % aqueous solution, for example available from McIntyre Group Limited under the trade mark MACKAM LAB. The amount stated refers to the as-supplied material.

Citric, malic, lactic acid and MBF all readily commercially available.

### Example 1

### Shampoo

| | |
|---|---|
| Softened Water | 70.96% |
| Xanthan Gum | 1.10% |
| Decyl Glucoside (50%) | 11.00% |
| Lauryl Betaine (30%) | 16.00% |
| Lactic Acid (80%) | 0.32% |
| Potassium Sorbate | 0.20% |
| Mixed Bioflavonoids | 0.20% |
| Citric Acid | 0.06% |
| Aloe Vera Powder | 0.16% |

20 g aliquots of the shampoo were inoculated individually using a single inoculum of bacteria or fungi. Inoculated samples were incubated at 25° C. and 1 gram removed after 0 hours, 2, 7, 14, 21 and 28 days. A plate count was performed on these samples with Letheen broth being used as the inactivator. Samples inoculated with bacteria were plated onto Tryptone Soya Agar and incubated at 33° C.±2° C. for 72 hours. Those samples inoculated with fungi were plated onto Sabouraud Dextrose Agar and incubated at 23° C.±2° C. for 5 days. The Total Viable Count per gram was then calculated using the dilution factor and recorded.

Inocula used were:
Aspergillus niger IMI 149007
Candida albicans NCPF 3179
Pseudomonas aeruginosa ATCC 9027
Staphylococcus aureus ATCC 6538

| | Inoculum | 0 | 2 | 7 | 14 | 28 |
|---|---|---|---|---|---|---|
| Culture | in sample | hour | days | days | days | days |
| A. niger | 1.42 × 10⁵ | 6.2 × 10⁵ | NT | NT | <20 | <20 |
| C. albicans | 1.5 × 10⁵ | 2.0 × 10⁵ | NT | NT | <20 | <20 |
| Ps. aeruginosa | 3.8 × 10⁶ | <20 | <20 | <20 | NT | <20 |
| S. aureus | 2.56 × 10⁶ | <20 | <20 | <20 | NT | NT |

NT in these examples = Not Tested

### Example 2

### Lotion Base

| | |
|---|---|
| Softened Water | 82.08% |
| Cetearyl Alcohol/ | 4.40% |
| Alkyl Glucoside | |
| Coconut Oil | 5.25% |
| Sunflower Oil | 5.25% |
| Mixed Tocopherols | 0.10% |
| Apricot Kernel Oil | 0.51% |
| Shea Butter | 0.99% |
| Xanthan Gum | 0.55% |
| Potassium Sorbate | 0.20% |
| Citric Acid | 0.25% |
| Mixed Bioflavonoids | 0.20% |
| Aloe Vera Powder | 0.22% |

Biocidal testing was carried out in a manner consistent with that described in Example 1. The results were as follows.

| | Inoculum | 0 | 2 | 7 | 14 | 28 |
|---|---|---|---|---|---|---|
| Culture | in sample | hour | days | days | days | days |
| A. niger | 1.42 × 10⁵ | 3 × 10⁵ | NT | NT | <20 | <20 |
| C. albicans | 1.5 × 10⁵ | 1.72 × 10⁵ | NT | NT | <20 | <20 |
| Ps. aeruginosa | 3.8 × 10⁶ | 3.8 × 10⁶ | <20 | <20 | NT | <20 |
| S. aureus | 2.56 × 10⁶ | 3.62 × 10⁶ | <20 | <20 | NT | NT |

### Example 3

### Baby Wash

| | |
|---|---|
| Softened Water | 75.99% |
| Betaine | 2.00% |
| Xanthan Gum | 1.00% |
| Decyl Glucoside | 8.10% |
| Lauryl Betaine | 11.40% |
| Potassium Sorbate | 0.20% |
| Lactic Acid | 0.30% |
| Citric Acid | 0.20% |
| Mixed Bioflavonoids | 0.20% |
| Aloe Vera Powder | 0.37% |
| Chamomile Oil | 0.10% |
| Fragrance | 0.14% |

Biocidal testing was carried out in a manner consistent with that described in Example 1. The results were as follows.

| | Inoculum | 0 | 2 | 7 | 14 | 28 |
|---|---|---|---|---|---|---|
| Culture | in sample | hour | days | days | days | days |
| A. niger | 1.42 × 10⁵ | 5.2 × 10⁵ | NT | NT | <20 | <20 |
| C. albicans | 1.5 × 10⁵ | 1.44 × 10⁵ | NT | NT | <20 | <20 |
| Ps. aeruginosa | 3.8 × 10⁶ | 5.6 × 10⁵ | <20 | <20 | NT | <20 |
| S. aureus | 2.56 × 10⁶ | <20 | <20 | <20 | NT | NT |

### Example 4

### Hair Conditioner

| | |
|---|---|
| Softened Water | 87.18% |
| Betaine (simple) | 2.00% |
| Cetearyl Alcohol/ | 3.50% |
| Alkyl Glucoside | |
| Xanthan Gum | 0.50% |
| Ceto-Stearyl Alcohol | 2.00% |
| Sunflower Oil | 0.75% |
| Olive Oil | 0.95% |
| Jojoba Oil | 0.10% |
| Shea Butter | 0.95% |
| Coconut Oil | 0.95% |
| Mixed Tocopherols | 0.05% |
| Potassium Sorbate | 0.20% |
| Lactic Acid | 0.08% |
| Citric Acid | 0.10% |
| Mixed Bioflavonoids | 0.20% |
| Chamomile Oil | 0.10% |
| Sweet Orange Oil | 0.14% |
| Grapefruit Oil | 0.07% |
| Lemon Oil | 0.07% |
| Aloe Vera powder | 0.11% |

Biocidal testing was carried out in a manner consistent with that described in Example 1. The results were as follows.

| | Inoculum | 0 | 2 | 7 | 14 | 28 |
|---|---|---|---|---|---|---|
| Culture | in sample | hour | days | days | days | days |
| A. niger | 1.42 × 10⁵ | 2.2 × 10⁵ | NT | NT | <20 | <20 |
| C. albicans | 1.5 × 10⁵ | 3.0 × 10⁵ | NT | NT | <20 | <20 |
| Ps. aeruginosa | 3.8 × 10⁶ | 5.2 × 10⁶ | <20 | <20 | NT | <20 |
| S. aureus | 2.56 × 10⁶ | 3.0 × 10⁶ | <20 | <20 | NT | <20 |

### Comments

From consideration of the results presented here, it is believed that the following factors are of importance in respect of antimicrobial activity:
the quinone/phenol equilibrium within the bioflavonoids. This is believed to be a function of pH
microorganism cell wall penetration (believed to be influenced by the surfactant)
isoelectric point of the betaine surfactant (the protonation state at various pHs). This is believed to be a function of pH
the presence of organic acid(s)
relative concentrations of ingredients
overall dilution.

1. According to a first aspect of the present invention there is provided a preservative composition to be added to a formulated product, the preservative composition comprising at least one flavonoid, and at least one antimicrobial auxiliary, wherein the preservative composition is acidic.

2. A composition as claimed in claim 1, comprising at least one flavonoid selected from flavonols, flavonons and flavons.

3. A composition as claimed in claim 1, comprising at least two flavonoids selected from flavonols, flavonons and flavons.

4. A composition as claimed in claim 1, comprising at least one organic acid selected from one or more of ascorbic acid, citric acid, malic acid and lactic acid.

5. A composition as claimed in claim 1, wherein the antimicrobial auxiliary comprises at least one betaine surfactant.

6. A composition as claimed in claim 1, wherein the antimicrobial auxiliary comprises sorbic acid and/or a salt thereof, preferably potassium sorbate.

7. A product to be preserved, the product comprising base components applicable to that product and preservative components comprising at least one flavonoid held under acidic conditions, together with at least one antimicrobial auxiliary.

8. A product as claimed in claim 7 wherein the product is a personal product such as a shampoo, hair conditioner, skin lotion, baby bath wash, moisturiser, medicinal cream, cosmetic product, laundry formulation, paint, oral care product, food preparation for humans, or animal feed.

9. A product as claimed in claim 7 wherein the composition of any of claims 1 to 6 is added as such, or the components of that composition are added separately, to said base components applicable to that product.

10. A product as claimed in claim 8, comprising from 0.00001 wt % up to 10 wt % bioflavonoid(s) (in total, when there is more than one).

11. A product as claimed in claim 7, which has antimicrobial activity preventing the formation of colonies, in the product, of one, two, three or four of these microbes: Pseudomonas aeruginosa, Escherichia coli, Aspergillus niger and Candida albicans.

12. A method of manufacturing the product of claim 7, comprising: blending at least one flavonoid, held under acidic conditions, and at least one antimicrobial auxiliary, into base components of said product.

13. The use of a composition of claim 1, or of its components separately, to impart antimicrobial activity to a formulated product.
